SEQUENCE LISTING (13 sequences)

SEQ ID NO: 1 - protein, 253 aa, Synthetic construct
EVQLVESGGGLIQPGRSLRLSCAASGITFDDAVMHWVRQAPGKGLEWVAGISSNSGYIGYADSVKGRFTISRDNAKNSLYLQMNRLRAEDTAVYYCVKGLYSNPRGGAFDIWGQGTMVTVSSASTGGGGSGGGGSGGGGSVHSSYVLTQPPSVSVAPGQTATITCGGNNIGTKSVHWYQQKPGQAPVLVVYADSDRPSGIPERVSGSNSGNTATLTISRVEAGDEADYYCQVWDSRSDHLWVFGGGTKLTVLG

SEQ ID NO: 2 - protein, 125 aa, Synthetic construct
EVQLVESGGGLIQPGRSLRLSCAASGITFDDAVMHWVRQAPGKGLEWVAGISSNSGYIGYADSVKGRFTISRDNAKNSLYLQMNRLRAEDTAVYYCVKGLYSNPRGGAFDIWGQGTMVTVSSAST

SEQ ID NO: 3 - protein, 9 aa, Synthetic construct
ITFDDAVMH

SEQ ID NO: 4 - protein, 6 aa, Synthetic construct
GISSNS

SEQ ID NO: 5 - protein, 15 aa, Synthetic construct
VKGLYSNPRGGAFDI

SEQ ID NO: 6 - protein, 113 aa, Synthetic construct
VHSSYVLTQPPSVSVAPGQTATITCGGNNIGTKSVHWYQQKPGQAPVLVVYADSDRPSGIPERVSGSNSGNTATLTISRVEAGDEADYYCQVWDSRSDHLWVFGGGTKLTVLG

SEQ ID NO: 7 - protein, 11 aa, Synthetic construct
GGNNIGTKSVH

SEQ ID NO: 8 - protein, 7 aa, Synthetic construct
ADSDRPS

SEQ ID NO: 9 - protein, 12 aa, Synthetic construct
QVWDSRSDHLWV

SEQ ID NO: 10 - protein, 510 aa, Synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVTSYLNAS

SEQ ID NO: 11 - protein, 788 aa, Synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVTSYLNASGGGGSGGGGSGGGGSGGGGSGGGGSEVQLVESGGGLIQPGRSLRLSCAASGITFDDAVMHWVRQAPGKGLEWVAGISSNSGYIGYADSVKGRFTISRDNAKNSLYLQMNRLRAEDTAVYYCVKGLYSNPRGGAFDIWGQGTMVTVSSASTGGGGSGGGGSGGGGSVHSSYVLTQPPSVSVAPGQTATITCGGNNIGTKSVHWYQQKPGQAPVLVVYADSDRPSGIPERVSGSNSGNTATLTISRVEAGDEADYYCQVWDSRSDHLWVFGGGTKLTVLG

SEQ ID NO: 12 - protein, 305 aa, Synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPC

SEQ ID NO: 13 - protein, 197 aa, Synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVTSYLNAS

(19) United States
(12) Patent Application Publication
(10) Pub. No.: US 2026/0193330 A1
(43) Pub. Date: Jul. 9, 2026

(54) METHODS FOR THE TREATMENT OF CANCER WITH RECOMBINANT IL-12 ALBUMIN BINDING DOMAIN FUSION PROTEINS

(71) Applicant: SONNET BIOTHERAPEUTICS, INC., Princeton, NJ (US)

(72) Inventors: John K. CINI, Blairstown, NJ (US); Richard T. KENNEY, Princeton, NJ (US)

(21) Appl. No.: 19/123,745

(22) PCT Filed: Nov. 1, 2023
(86) PCT No.: PCT/US2023/078366

§ 371 Date: Apr. 23, 2025

Related U.S. Application Data
(60) Provisional application No. 63/421,846, filed on Nov. 2, 2022.

(51) Int. Cl. C07K 16/18; A61K 9/00; A61K 38/20; A61K 45/06; A61K 47/68; A61P 35/00

(52) U.S. Cl. CPC C07K 16/18; A61K 9/0019; A61K 38/208; A61K 45/06; A61K 47/6813; A61P 35/00; C07K 2317/56; C07K 2317/622; C07K 2319/00

(57) ABSTRACT

The present disclosure provides methods for treating a cancer such as an advanced solid tumor in a subject in need thereof by administering to the subject in need thereof an effective first dose of an IL-12 albumin binding domain (ABD) fusion protein and an effective second dose of the IL-12 ABD fusion protein every three weeks or four weeks after the first dose, wherein the second dose is greater than the first dose.

### CROSS-REFERENCE TO RELATED APPLICATIONS

This application claims priority to U.S. Provisional Patent Application No. 63/421,846, filed on Nov. 2, 2022, which is hereby incorporated by reference in its entirety.

### FIELD

The present disclosure generally provides methods for treating a cancer with an IL-12 albumin binding domain (ABD) fusion protein.

### SEQUENCE LISTING

The instant application contains a Sequence Listing which has been submitted electronically in XML format and is hereby incorporated by reference in its entirety. Said XML file is named 116076_5020_WO_Sequence_Listing.xml, was created on Oct. 30, 2023, and is 15000 bytes in size.

### BACKGROUND

IL-12 is capable of mediating immune effector functions in a manner compatible to enhancing pro-inflammatory, endogenous anti-tumor immune response. (See, e.g., Boggio et al., J Exp Med 188:589-96 (1998); Cavallo et al., Cancer Res 59:414-21 (1999); Yu et al. Int Immunol 8:855-65 (1996); Nastala et al., J Immunol 153:1697:706 (1994); Brunda et al., J Exp Med 178:1223-30 (1993). IL-12 is known to induce an inflammatory Th1 CD4+ T-cell response as well as enhance CD8+ T-cell cytotoxicity. Studies have also shown that T-cell secretion of IFNγ mediated by IL-12 may reverse T-cell anergy and confer effector T-cell resistance to immune suppressive regulatory T-cells. The ability of IL-12 to not only activate the adaptive and the innate immune systems, but also further modulate the otherwise immune-hostile tumor microenvironment makes IL-12 an ideal candidate for tumor immunotherapy.

Serum albumin possesses a long half-life in the range of 2-4 weeks due to recycling through the neonatal Fc receptor (FcRn). Albumin is taken up by endothelial cells through macropinocytosis and binds to the FcRn in a pH-dependent manner in the acidic environment of the early endosome. Albumin-FcRn binding diverts albumin molecules from degradation in the lysosomal compartment and redirects the albumin molecules to the plasma membrane, where they are released back into the blood plasma due to the neutral pH.

Albumin binding domain (ABD) fusion proteins as described in U.S. Pat. No. 11,028,166 are useful for extending the half-lives of biologics (e.g., interleukins and antibodies). Such ABDs do not compete with FcRN for albumin binding and bind albumin at a pH range that allows for the ABD to also undergo FcRn-driven endosomal albumin recycling when bound to albumin. As such, biologics that include the ABD are capable of evading lysosomal degradation using the albumin-FcRn pathway and, consequently, exhibit longer serum half-lives than counterparts lacking ABDs. Moreover, such ABD containing therapeutics advantageously localize to tumors, which are known to contain high levels of serum albumin. Thus, such ABD containing therapeutics are particularly useful for the treatment of cancers.

### SUMMARY

Provided herein are methods for treating a cancer with an IL-12 albumin binding domain (ABD) fusion protein.

In one aspect, the present disclosure provides a method for treating a cancer in a subject in need thereof, the method comprising: administering to the subject in need thereof an effective first dose of an IL-12 albumin binding domain (ABD) fusion protein and an effective second dose of the IL-12 ABD fusion protein every three weeks or four weeks after the first dose, wherein the second dose is greater than the first dose.

In one aspect, the present disclosure provides a method of achieving a clinical response or a partial clinical response of a solid tumor in a human patient, the method comprising: administering to the human patient an effective first dose of an IL-12 albumin binding domain (ABD) fusion protein and an effective second dose of the IL-12 ABD fusion protein every two weeks, three weeks, or four weeks after the first dose, wherein the second dose is greater than the first dose.

In one embodiment, the effective second and subsequent doses of the IL-12 ABD fusion protein is administered every three weeks after the first dose.

In one embodiment, the effective second and subsequent doses of the IL-12 ABD fusion protein is administered every four weeks after the first dose.

In one embodiment, the first dose is about 25 ng/kg to about 750 ng/kg.

In one embodiment, the first dose is about 150 ng/kg.

In one embodiment, the second and subsequent doses are about 50 ng/kg to about 1.5 μg/kg.

In one embodiment, the second and subsequent doses are about 300 ng/kg.

In one embodiment, the second and subsequent doses are about 540 ng/kg.

In one embodiment, the second and subsequent doses are about 750 ng/kg.

In one embodiment, the first dose amount and the second and subsequent dose amounts are administered intravenously.

In one embodiment, the first dose amount and the second and subsequent dose amounts are administered subcutaneously.

In one embodiment, the albumin binding domain of the IL-12 ABD fusion protein comprises a variable heavy domain comprising SEQ ID NO:2 or a variant thereof, and a variable light domain comprising SEQ ID NO:6 or a variant thereof.

In one embodiment, the albumin binding domain is an scFv that comprises the amino acid sequence of SEQ ID NO: 1 or a variant thereof.

In one embodiment, the IL-12 of the IL-12 fusion protein is a single chain IL-12 comprising a p35 subunit covalently attached to a p40 subunit.

In one embodiment, the p40 subunit has the amino acid sequence of SEQ ID NO:12 or a variant thereof.

In one embodiment, the p35 subunit has the amino acid sequence of SEQ ID NO:13 or a variant thereof.

In one embodiment, the single chain IL-12 has the amino acid sequence of SEQ ID NO: 10.

In one embodiment, the IL-12 ABD fusion protein has the amino acid sequence of SEQ ID NO: 11.

In one embodiment, the subject is a human patient.

In one embodiment, the human patient is an adult human patient.

In one embodiment, the cancer is a solid tumor.

In one embodiment, the solid tumor is a solid epithelial or mesenchymal tumor.

In one embodiment, the method further comprises the step of administering a checkpoint inhibitor to the subject in need thereof.

In one embodiment, the subject in need thereof previously received standard of care therapy or is not a candidate for standard of care therapy.

In an embodiment, the solid tumor is an advanced solid tumor.

### BRIEF DESCRIPTION OF THE DRAWINGS

FIG. 1 depicts an exemplary IL-12 albumin binding domain (ABD) fusion protein that can be administered. The IL-12 depicted is a single chain IL-12 molecule, wherein the IL-12A (p35) and IL-12B (p40) components are attached using a polypeptide linker.

FIG. 2A-FIG. 2B depicts the albumin binding domain component of the IL-12 albumin binding domain fusion proteins that can be used in the IL-12 ABD fusion proteins administered using the subject methods provided herein.

FIG. 3 are a series of graphs depicting the cytokine response in Cohorts that received 50 ng/kg, 100 ng/kg, and 150 ng/kg of a IL-12 ABD fusion protein, SON-1010, respectively.

FIG. 4 provides baseline and follow-up results from patients in Cohorts M1, M2, and M3 of the study indicated in Example 1.

### DETAILED DESCRIPTION

### I. Overview

The present disclosure provides methods for treating a cancer such as an advanced solid tumor in a subject in need thereof by administering to the subject in need thereof an effective first dose of an IL-12 albumin binding domain (ABD) fusion protein and an effective second dose of the IL-12 ABD fusion protein every three weeks or four weeks after the first dose, wherein the second dose is greater than the first dose.

### II. Definitions

In order that the application may be more completely understood, several definitions are set forth below. Such definitions are meant to encompass grammatical equivalents.

The term “a” or “an” refers to one or more of that entity, i.e. can refer to a plural referent. As such, the terms “a” or “an”, “one or more” and “at least one” are used interchangeably herein. In addition, reference to “an element” by the indefinite article “a” or “an” does not exclude the possibility that more than one of the elements is present, unless the context clearly requires that there is one and only one of the elements.

It is noted that the claims may be drafted to exclude any optional element. As such, this statement is intended to serve as an antecedent basis for use of such exclusive terminology as “solely,” “only,” and the like in connection with the recitation of claim elements or use of a “negative” limitation. As will be apparent to those of skill in the art upon reading this disclosure, each of the individual embodiments described and illustrated herein has discrete components and features readily separated from or combined with the features of any of the other several embodiments without departing from the scope or spirit of the invention. Any recited method may be carried out in the order of events recited or in any other order that is logically possible. Although any methods and materials similar or equivalent to those described herein may also be used in the practice or testing of the invention, representative illustrative methods and materials are now described.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

Unless specifically stated or obvious from context, as used herein, the term “about” is understood as within a range of normal tolerance in the art, for example within 2 standard deviations of the mean. About can be understood as within 50%, 45%, 40%, 35%, 30%, 25%, 20%, 15%, 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Before the invention is further described, it is to be understood that this invention is not limited to particular embodiments described, as such may, of course, vary. It is also to be understood that the terminology used herein is for describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are encompassed within the invention, subject to any specifically excluded limit in the stated range. Where the stated range includes one or both of the limits, ranges excluding either or both of those included limits are also included in the invention. Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number, which, in the context presented, provides the substantial equivalent of the specifically recited number.

All publications, patents, and patent applications cited in this specification are incorporated herein by reference to the same extent as if each individual publication, patent, or patent application were specifically and individually indicated to be incorporated by reference. Furthermore, each cited publication, patent, or patent application is incorporated herein by reference to disclose and describe the subject matter in connection with which the publications are cited. The citation of any publication is for its disclosure prior to the filing date and should not be construed as an admission that the invention described herein is not entitled to antedate such publication by virtue of prior invention. Further, the dates of publication provided might be different from the actual publication dates, which may need to be independently confirmed.

### III. IL-12 Albumin Binding Domain Fusion Protein

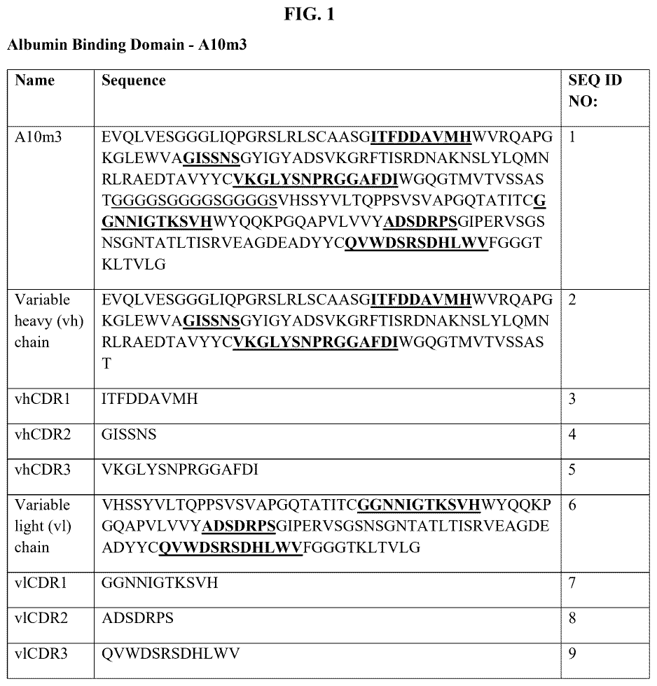

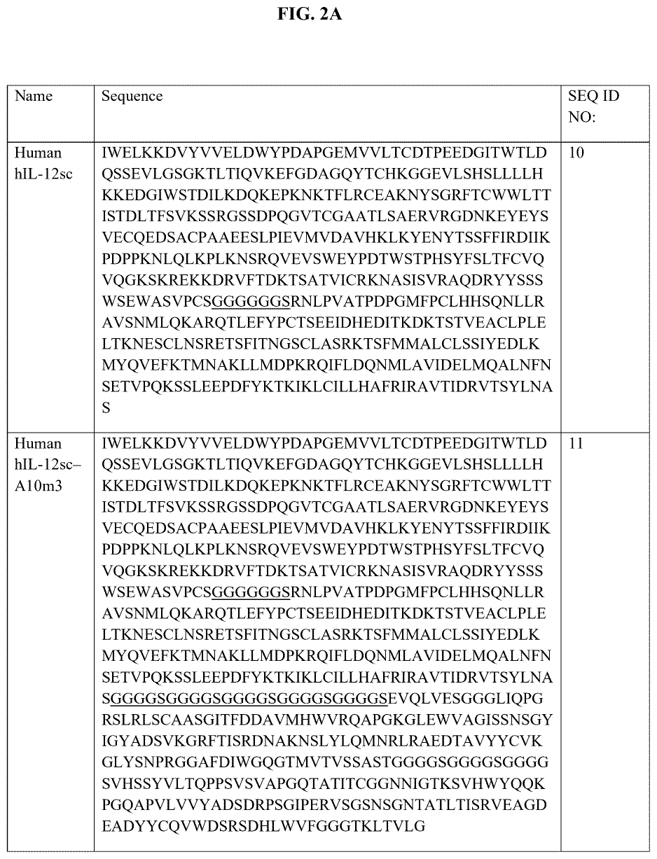

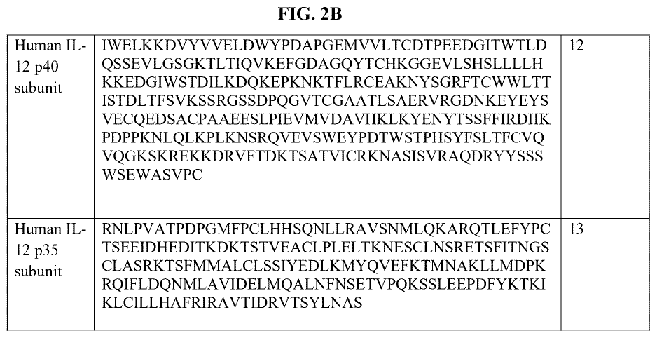

In embodiments, the subject methods described herein are useful for producing IL-12 albumin binding domain (ABD) fusion proteins. Such IL-12 ABD fusion protein find use, for example, in the treatment of cancers. In some embodiments, the IL-12 albumin binding domain fusion protein includes an albumin binding domain that includes an antibody variable heavy chain domain that includes a vhCDR1 having the amino acid sequence of SEQ ID NO:3, a vhCDR2 having the amino acid sequence of SEQ ID NO:4, and a vhCDR3 having the amino acid sequence of SEQ ID NO:5 (see FIG. 1). In some embodiments, the albumin binding domain includes a variable light chain domain that includes a vlCDRI having the amino acid sequence of SEQ ID NO:7, a vICDR2 having the amino acid sequence of SEQ ID NO:8, and a vlCDR3 having the amino acid sequence of SEQ ID NO:9. In embodiments, the albumin binding domain includes the variable heavy chain having the amino acid sequence of SEQ ID NO: 2 and/or a variable light chain having the amino acid sequence of SEQ ID NO:6. In embodiments, the albumin binding domain includes the variable heavy chain having the amino acid sequence of SEQ ID NO:2 and a variable light chain having the amino acid sequence of SEQ ID NO:6. In embodiments, the albumin binding domain of the IL-12 albumin binding domain fusion protein is an scFv that has the amino acid sequence of the A10m3 (SEQ ID NO: 1).

In embodiments, the IL-12 albumin binding domain fusion protein includes an albumin binding domain that is a variant of the A10m3 albumin binding domain depicted in FIG. 1. In exemplary embodiments, the albumin binding domain of the IL-12 albumin binding domain fusion protein includes a set of 6 CDRs with 1, 2, 3, 4, 5, 6, 7, 8, 9, 10 amino acid modifications as compared to the 6 CDRs of A10m3, as depicted in FIG. 1. In embodiments, the albumin binding domain of the IL-12 albumin binding domain fusion protein includes 6 CDRs that are at least 90, 95, 97, 98 or 99% identical to the 6 CDRs of A10m3 (see FIG. 1). In embodiments, the albumin binding domain includes a VH domain and/or VL domain that has from 1, 2, 3, 4, 5, 6, 7, 8, 9 or 10 amino acid changes from a VH and/or VL domain of A10m3, as depicted in FIG. 1. In embodiments, the albumin binding domain includes a VH domain and/or VL domain that is at least 90, 95, 97, 98 or 99% identical to the VH and/or VL of A10m3, as depicted in FIG. 1. In certain embodiments, the variant of the A10m3 albumin binding domain is capable of binding human serum albumin, as measured by at least one of a Biacore, surface plasmon resonance (SPR), flow cytometry, and/or BLI (biolayer interferometry, e.g., Octet assay) assay, with the latter finding particular use in many embodiments.

In certain embodiments, the IL-12 is a single chain IL-12 polypeptide comprising an IL-12 p35 subunit attached to an IL-12 p40 subunit. In embodiments, the IL-12 single chain polypeptides advantageously retain one or more of the biological activities of wildtype IL-12. In some embodiments, the single chain IL-12 polypeptide described herein is according to the formula, from N-terminus to C-terminus, (p40)-(L)-(p35), wherein “p40” is an IL-12 p40 subunit, “p35” is IL-12 p35 subunit and L is a linker. In other embodiments, the single chain IL-12 is according to the formula from N-terminus to C-terminus, (p35)-(L)-(p40). Any suitable linker can be used in the single chain IL-12 polypeptide. Suitable linkers can include, for example, linkers having the amino acid sequence (GGGGS)ₓ wherein x is an integer from 1-10. Other suitable linkers include, for example, the amino acid sequence GGGGGGS. Exemplary single chain IL-12 linkers than can be used with the subject single chain IL-12 polypeptides are also described in Lieschke et al., Nature Biotechnology 15:35-40 (1997), which is incorporated herein in its entirety by reference and particularly for its teaching of IL-12 polypeptide linkers.

In some embodiments, the IL-12 of the IL-12 albumin binding domain fusion protein includes a human p40 subunit having the amino acid sequence of SEQ ID NO:12. In some embodiments, the IL-12 includes a variant human p40 subunit that has from 1, 2, 3, 4, 5, 6, 7, 8, 9 or 10 amino acid changes as compared to SEQ ID NO:12. In embodiments, the IL-12 includes a variant human p40 that is at least 90, 95, 97, 98 or 99% identical to SEQ ID NO: 12.

In some embodiments, the IL-12 of the IL-12 albumin binding domain fusion protein includes a human p35 subunit having the amino acid sequence of SEQ ID NO:13. In some embodiments, the IL-12 includes a variant human p35 subunit that has from 1, 2, 3, 4, 5, 6, 7, 8, 9 or 10 amino acid changes as compared to SEQ ID NO:13. In embodiments, the IL-12 includes a variant human p40 that is at least 90, 95, 97, 98 or 99% identical to SEQ ID NO:13.

TABLE 1

| | | SEQ ID |
|---|---|---|
| Name | Sequence | NO: |
| A10m3 | EVQLVESGGGLIQPGRSLRLSCAASGITFDDAVMHWVRQAPG | 1 |
| | KGLEWVAGISSNSGYIGYADSVKGRFTISRDNAKNSLYLQMN | |
| | RLRAEDTAVYYCVKGLYSNPRGGAFDIWGQGTMVTVSSAST | |
| | GGGGSGGGGSGGGGSVHSSYVLTQPPSVSVAPGQTATITCGG | |
| | NNIGTKSVHWYQQKPGQAPVLVVYADSDRPSGIPERVSGSNS | |
| | GNTATLTISRVEAGDEADYYCQVWDSRSDHLWVFGGGTKLT | |
| | VLG | |
| A10m3 | EVQLVESGGGLIQPGRSLRLSCAASGITFDDAVMHWVRQAPG | 2 |
| Variable | KGLEWVAGISSNSGYIGYADSVKGRFTISRDNAKNSLYLQMN | |
| heavy (vh) | RLRAEDTAVYYCVKGLYSNPRGGAFDIWGQGTMVTVSSAST | |
| chain | | |
| A10m3 | ITFDDAVMH | 3 |
| vhCDR1 | | |
| A10m3 | GISSNS | 4 |
| vhCDR2 | | |
| A10m3 | VKGLYSNPRGGAFDI | 5 |
| vhCDR3 | | |
| A10m3 | VHSSYVLTQPPSVSVAPGQTATITCGGNNIGTKSVHWYQQKP | 6 |
| Variable | GQAPVLVVYADSDRPSGIPERVSGSNSGNTATLTISRVEAGDE | |
| light (vl) | ADYYCQVWDSRSDHLWVFGGGTKLTVLG | |
| chain | | |
| A10m3 | GGNNIGTKSVH | 7 |
| VICDR1 | | |
| A10m3 | ADSDRPS | 8 |
| VICDR2 | | |
| A10m3 | QVWDSRSDHLWV | 9 |
| VICDR3 | | |
| Human | IWELKKDVYVVELDWYPDAPGEMVVLTCDTPEEDGITWT | 10 |
| hIL-12sc | LDQSSEVLGSGKTLTIQVKEFGDAGQYTCHKGGEVLSHSL | |
| | LLLHKKEDGIWSTDILKDQKEPKNKTFLRCEAKNYSGRFT | |
| | CWWLTTISTDLTFSVKSSRGSSDPQGVTCGAATLSAERVR | |
| | GDNKEYEYSVECQEDSACPAAEESLPIEVMVDAVHKLKYE | |
| | NYTSSFFIRDIIKPDPPKNLQLKPLKNSRQVEVSWEYPDTW | |
| | STPHSYFSLTFCVQVQGKSKREKKDRVFTDKTSATVICRK | |
| | NASISVRAQDRYYSSSWSEWASVPCSGGGGGGSRNLPVATP | |
| | DPGMFPCLHHSQNLLRAVSNMLQKARQTLEFYPCTSEEIDHE | |
| | DITKDKTSTVEACLPLELTKNESCLNSRETSFITNGSCLASR | |
| | KTSFMMALCLSSIYEDLKMYQVEFKTMNAKLLMDPKRQIFLDQ | |
| | NMLAVIDELMQALNFNSETVPQKSSLEEPDFYKTKIKLCILLH | |
| | AFRIRAVTIDRVTSYLNAS | |
| Human | IWELKKDVYVVELDWYPDAPGEMVVLTCDTPEEDGITWT | 11 |
| hIL-12sc- | LDQSSEVLGSGKTLTIQVKEFGDAGQYTCHKGGEVLSHSL | |
| A10m3 | LLLHKKEDGIWSTDILKDQKEPKNKTFLRCEAKNYSGRFT | |
| | CWWLTTISTDLTFSVKSSRGSSDPQGVTCGAATLSAERVR | |
| | GDNKEYEYSVECQEDSACPAAEESLPIEVMVDAVHKLKYE | |
| | NYTSSFFIRDIIKPDPPKNLQLKPLKNSRQVEVSWEYPDTW | |
| | STPHSYFSLTFCVQVQGKSKREKKDRVFTDKTSATVICRK | |
| | NASISVRAQDRYYSSSWSEWASVPCSGGGGGGSRNLPVATP | |
| | DPGMFPCLHHSQNLLRAVSNMLQKARQTLEFYPCTSEEIDHE | |
| | DITKDKTSTVEACLPLELTKNESCLNSRETSFITNGSCLASR | |
| | KTSFMMALCLSSIYEDLKMYQVEFKTMNAKLLMDPKRQIFLDQ | |
| | NMLAVIDELMQALNFNSETVPQKSSLEEPDFYKTKIKLCILLH | |
| | AFRIRAVTIDRVTSYLNASGGGGSGGGGSGGGGSGGGGSGGG | |
| | GSEVQLVESGGGLIQPGRSLRLSCAASGITFDDAVMHWVRQA | |
| | PGKGLEWVAGISSNSGYIGYADSVKGRFTISRDNAKNSLYLQ | |
| | MNRLRAEDTAVYYCVKGLYSNPRGGAFDIWGQGTMVTVSSA | |
| | STGGGGSGGGGSGGGGSVHSSYVLTQPPSVSVAPGQTATITC | |
| | GGNNIGTKSVHWYQQKPGQAPVLVVYADSDRPSGIPERVSGS | |
| | NSGNTATLTISRVEAGDEADYYCQVWDSRSDHLWVFGGGTK | |
| | LTVLG | |
| Human IL- | IWELKKDVYVVELDWYPDAPGEMVVLTCDTPEEDGITWTLD | 12 |
| 12 p40 | QSSEVLGSGKTLTIQVKEFGDAGQYTCHKGGEVLSHSLLLLH | |
| subunit | KKEDGIWSTDILKDQKEPKNKTFLRCEAKNYSGRFTCWWLTT | |
| | ISTDLTFSVKSSRGSSDPQGVTCGAATLSAERVRGDNKEYEY | |
| | SVECQEDSACPAAEESLPIEVMVDAVHKLKYENYTSSFFIRD | |
| | IIKPDPPKNLQLKPLKNSRQVEVSWEYPDTWSTPHSYFSLTF | |
| | CVQVQGKSKREKKDRVFTDKTSATVICRKNASISVRAQDRYY | |
| | SSSWSEWASVPC | |
| Human IL- | RNLPVATPDPGMFPCLHHSQNLLRAVSNMLQKARQTLEFYPC | 13 |
| 12 p35 | TSEEIDHEDITKDKTSTVEACLPLELTKNESCLNSRETSFIT | |
| subunit | NGSCLASRKTSFMMALCLSSIYEDLKMYQVEFKTMNAKLLMD | |
| | PKRQIFLDQNMLAVIDELMQALNFNSETVPQKSSLEEPDFYK | |
| | TKIKLCILLHAFRIRAVTIDRVTSYLNAS | |

### IV. Methods of Treating Cancer

Provided herein are methods for the treatment of cancer solid tumor (e.g., a solid epithelial or mesenchymal tumor) in a subject in need thereof such as a human patient. Such methods may comprise administering (e.g., subcutaneously or intravenously) to the subject in need thereof an effective first dose (a desensitization dose) of an IL-12 albumin binding domain (ABD) fusion protein as disclosed herein (e.g., IL12-FHAB or SON-1010) and an effective second dose (a maintenance dose) of the IL-12 ABD fusion protein as disclosed herein every two to six weeks after the first dose, wherein the second dose is greater than the first dose. In some embodiments, the first dose and the second dose are clinically effective doses. The subject in need thereof may have previously received a standard of care therapy or is not a candidate for a standard of care therapy. In some embodiments, the subject in need thereof may additionally be administered an immune checkpoint inhibitor concurrently or separately with the first and/or second dose. In some embodiments, the IL-12 albumin binding domain (ABD) fusion protein is administered subcutaneously into the deltoid muscle of the upper arm.

The first dose may be administered on day 0 followed by the second dose every three weeks on about day 21, about day 42, about day 63 and so on. Alternatively, the first dose may be administered on day 0 followed by the second dose every four weeks on about day 28, about day 56, about day 84 and so on.

In some embodiments, the effective second dose is administered every two weeks after the first dose. In other embodiments, the effective second dose is administered every three weeks after the first dose. In another embodiment, the effective second dose is administered every four weeks after the first dose. In other embodiments, the effective second dose is administered every five weeks after the first dose. In another embodiment, the effective second dose is administered every six weeks after the first dose.

The first dose administered in the methods disclosed herein may be about 25 ng/kg to about 750 ng/kg including, for example, about 25 ng/kg, about 30 ng/kg, about 35 ng/kg, about 40 ng/kg, about 45 ng/kg, about 50 ng/kg, about 55 ng/kg, about 60 ng/kg, about 65 ng/kg, about 70 ng/kg, about 75 ng/kg, about 80 ng/kg, about 85 ng/kg, about 90 ng/kg, about 95 ng/kg, about 100 ng/kg, about 105 ng/kg, about 110 ng/kg, about 115 ng/kg, about 120 ng/kg, about 125 ng/kg, about 130 ng/kg, about 135 ng/kg, about 140 ng/kg, about 145 ng/kg, about 150 ng/kg, about 155 ng/kg, about 160 ng/kg, about 165 ng/kg, about 170 ng/kg, about 175 ng/kg, about 180 ng/kg, about 185 ng/kg, about 190 ng/kg, about 195 ng/kg, about 200 ng/kg, about 205 ng/kg, about 210 ng/kg, about 215 ng/kg, about 220 ng/kg, about 225 ng/kg, about 230 ng/kg, about 235 ng/kg, about 240 ng/kg, about 245 ng/kg, about 250 ng/kg, about 255 ng/kg, about 260 ng/kg, about 265 ng/kg, about 270 ng/kg, about 275 ng/kg, about 280 ng/kg, about 285 ng/kg, about 290 ng/kg, about 295 ng/kg, about 300 ng/kg, about 305 ng/kg, about 310 ng/kg, about 315 ng/kg, about 320 ng/kg, about 325 ng/kg, about 330 ng/kg, about 335 ng/kg, about 340 ng/kg, about 345 ng/kg, about 350 ng/kg, about 355 ng/kg, about 360 ng/kg, about 365 ng/kg, about 370 ng/kg, about 375 ng/kg, about 380 ng/kg, about 385 ng/kg, about 390 ng/kg, about 395 ng/kg, about 400 ng/kg, about 405 ng/kg, about 410 ng/kg, about 415 ng/kg, about 420 ng/kg, about 425 ng/kg, about 430 ng/kg, about 435 ng/kg, about 440 ng/kg, about 445 ng/kg, about 450 ng/kg, about 455 ng/kg, about 460 ng/kg, about 465 ng/kg, about 470 ng/kg, about 475 ng/kg, about 480 ng/kg, about 485 ng/kg, about 490 ng/kg, about 495 ng/kg, about 500 ng/kg, about 505 ng/kg, about 510 ng/kg, about 515 ng/kg, about 520 ng/kg, about 525 ng/kg, about 530 ng/kg, about 535 ng/kg, about 540 ng/kg, about 545 ng/kg, about 550 ng/kg, about 555 ng/kg, about 560 ng/kg, about 565 ng/kg, about 570 ng/kg, about 575 ng/kg, about 580 ng/kg, about 585 ng/kg, about 590 ng/kg, about 595 ng/kg, about 600 ng/kg, about 605 ng/kg, about 610 ng/kg, about 615 ng/kg, about 620 ng/kg, about 625 ng/kg, about 630 ng/kg, about 635 ng/kg, about 640 ng/kg, about 645 ng/kg, about 650 ng/kg, about 655 ng/kg, about 660 ng/kg, about 665 ng/kg, about 670 ng/kg, about 675 ng/kg, about 680 ng/kg, about 685 ng/kg, about 690 ng/kg, about 695 ng/kg, about 700 ng/kg, about 705 ng/kg, about 710 ng/kg, about 715 ng/kg, about 720 ng/kg, about 725 ng/kg, about 730 ng/kg, about 735 ng/kg, about 740 ng/kg, about 745 ng/kg, and about 750 ng/kg. In an embodiment, the first dose is about 150 ng/kg.

The second dose administered in the methods disclosed herein may be about 50 ng/kg to about 1.5 μg/kg including, for example, about 50 ng/kg, about 55 ng/kg, about 60 ng/kg, about 65 ng/kg, about 70 ng/kg, about 75 ng/kg, about 80 ng/kg, about 85 ng/kg, about 90 ng/kg, about 95 ng/kg, about 100 ng/kg, about 105 ng/kg, about 110 ng/kg, about 115 ng/kg, about 120 ng/kg, about 125 ng/kg, about 130 ng/kg, about 135 ng/kg, about 140 ng/kg, about 145 ng/kg, about 150 ng/kg, about 155 ng/kg, about 160 ng/kg, about 165 ng/kg, about 170 ng/kg, about 175 ng/kg, about 180 ng/kg, about 185 ng/kg, about 190 ng/kg, about 195 ng/kg, about 200 ng/kg, about 205 ng/kg, about 210 ng/kg, about 215 ng/kg, about 220 ng/kg, about 225 ng/kg, about 230 ng/kg, about 235 ng/kg, about 240 ng/kg, about 245 ng/kg, about 250 ng/kg, about 255 ng/kg, about 260 ng/kg, about 265 ng/kg, about 270 ng/kg, about 275 ng/kg, about 280 ng/kg, about 285 ng/kg, about 290 ng/kg, about 295 ng/kg, about 300 ng/kg, about 305 ng/kg, about 310 ng/kg, about 315 ng/kg, about 320 ng/kg, about 325 ng/kg, about 330 ng/kg, about 335 ng/kg, about 340 ng/kg, about 345 ng/kg, about 350 ng/kg, about 355 ng/kg, about 360 ng/kg, about 365 ng/kg, about 370 ng/kg, about 375 ng/kg, about 380 ng/kg, about 385 ng/kg, about 390 ng/kg, about 395 ng/kg, about 400 ng/kg, about 405 ng/kg, about 410 ng/kg, about 415 ng/kg, about 420 ng/kg, about 425 ng/kg, about 430 ng/kg, about 435 ng/kg, about 440 ng/kg, about 445 ng/kg, about 450 ng/kg, about 455 ng/kg, about 460 ng/kg, about 465 ng/kg, about 470 ng/kg, about 475 ng/kg, about 480 ng/kg, about 485 ng/kg, about 490 ng/kg, about 495 ng/kg, about 500 ng/kg, about 505 ng/kg, about 510 ng/kg, about 515 ng/kg, about 520 ng/kg, about 525 ng/kg, about 530 ng/kg, about 535 ng/kg, about 540 ng/kg, about 545 ng/kg, about 550 ng/kg, about 555 ng/kg, about 560 ng/kg, about 565 ng/kg, about 570 ng/kg, about 575 ng/kg, about 580 ng/kg, about 585 ng/kg, about 590 ng/kg, about 595 ng/kg, about 600 ng/kg, about 605 ng/kg, about 610 ng/kg, about 615 ng/kg, about 620 ng/kg, about 625 ng/kg, about 630 ng/kg, about 635 ng/kg, about 640 ng/kg, about 645 ng/kg, about 650 ng/kg, about 655 ng/kg, about 660 ng/kg, about 665 ng/kg, about 670 ng/kg, about 675 ng/kg, about 680 ng/kg, about 685 ng/kg, about 690 ng/kg, about 695 ng/kg, about 700 ng/kg, about 705 ng/kg, about 710 ng/kg, about 715 ng/kg, about 720 ng/kg, about 725 ng/kg, about 730 ng/kg, about 735 ng/kg, about 740 ng/kg, about 745 ng/kg, about 750 ng/kg, about 755 ng/kg, about 760 ng/kg, about 765 ng/kg, about 770 ng/kg, about 775 ng/kg, about 780 ng/kg, about 785 ng/kg, about 790 ng/kg, about 795 ng/kg, about 800 ng/kg, about 805 ng/kg, about 810 ng/kg, about 815 ng/kg, about 820 ng/kg, about 825 ng/kg, about 830 ng/kg, about 835 ng/kg, about 840 ng/kg, about 845 ng/kg, about 850 ng/kg, about 855 ng/kg, about 860 ng/kg, about 865 ng/kg, about 870 ng/kg, about 875 ng/kg, about 880 ng/kg, about 885 ng/kg, about 890 ng/kg, about 895 ng/kg, about 900 ng/kg, about 905 ng/kg, about 910 ng/kg, about 915 ng/kg, about 920 ng/kg, about 925 ng/kg, about 930 ng/kg, about 935 ng/kg, about 940 ng/kg, about 945 ng/kg, about 950 ng/kg, about 955 ng/kg, about 960 ng/kg, about 965 ng/kg, about 970 ng/kg, about 975 ng/kg, about 980 ng/kg, about 985 ng/kg, about 990 ng/kg, about 995 ng/kg, about 1.00 μg/kg, about 1.05 μg/kg, about 1.1 μg/kg, about 1.15 μg/kg, about 1.2 μg/kg, about 1.25 μg/kg, about 1.3 μg/kg, about 1.35 μg/kg, about 1.4 μg/kg, about 1.45 μg/kg, and about 1.5 μg/kg.

In an embodiment, the second dose is about 300 ng/kg. In an embodiment, the second dose is about 540 ng/kg. In an embodiment, the second dose is about 750 ng/kg.

The second and subsequent doses (maintenance dose) may be administered to the subject in need thereof for 2 months, 3 months, 4 months, 5 months, 6 months, 7, months, 8 months, 9 months, 10 months, 11 months, 12 months, 13 months, 14 months, 15 months 16 months, 17 months, 18 months, 19 months, 20 months, 21 months, 22 months, 23 months, 24 months, or longer.

The dosing regimen disclosed herein advantageously achieve a clinical response or a partial clinical response of a solid tumor in a human patient. Response of a solid tumor may be assessed using RECIST v1.1 criteria. A complete response (CR) is characterized by a disappearance of all target lesions or continued no evidence of disease. Additionally, any pathological lymph nodes (whether target or nontarget) must have reduction in short axis to <10 mm. A partial response (PR) is characterized by at least a 30% decrease in the sum of the diameters of target lesions, taking as reference the baseline sum diameters.

Additionally, in some embodiments, the dosing regimen disclosed herein may surprisingly achieve a clinical response of a nontarget lesion. A complete response (CR) of a nontarget lesion is characterized by a disappearance of all nontarget lesions and normalization of tumor marker level. Additionally, all lymph nodes must be nonpathological in size (<10 mm short axis).

### V. Pharmaceutical Formulations

In another aspect, provided herein is a therapeutic composition comprising the IL-12 ABD fusion proteins and a carrier. Subject therapeutic compositions used in the practice of the foregoing methods can be formulated into pharmaceutical compositions comprising a carrier suitable for the desired delivery method. Suitable carriers include any material that when combined with the therapeutic composition retains the anti-tumor function of the therapeutic composition and is generally non-reactive with the patient's immune system. Examples include, but are not limited to, any of a number of standard pharmaceutical carriers such as sterile phosphate buffered saline solutions, bacteriostatic water, and the like (see, generally, Remington's Pharmaceutical Sciences 16th Edition, A. Osal., Ed., 1980).

Formulations of the albumin binding domain (ABD) polypeptide used in accordance with the present invention are prepared for storage by mixing an ABD polypeptide having the desired degree of purity with optional pharmaceutically acceptable carriers, excipients or

stabilizers (Remington's Pharmaceutical Sciences 16th edition, Osol, A. Ed. [1980]), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA or DPTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The formulation herein may also contain more than one active compound as necessary for the particular indication being treated, preferably those with complementary activities that do not adversely affect each other. For example, it may be desirable to provide multivalent interleukin polypeptide with other specificities. Alternatively, or in addition, the composition may comprise a cytotoxic agent, cytokine, growth inhibitory agent and/or small molecule antagonist. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for in vivo administration should be sterile, or nearly so. This is readily accomplished by filtration through sterile filtration membranes.

Sustained-release preparations may be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and .gamma ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

When encapsulated albumin binding domain polypeptides remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

### EXAMPLES

Examples are provided below to illustrate the present invention. These examples are not meant to constrain the present invention to any particular application or theory of operation.

### Example 1: a Phase 1, Dose-Escalation Study to Assess the Safety, Tolerability, Pharmacokinetics, and Pharmacodynamics of SON-1010 (IL-12-FHAB) in Adult Patients with Advanced Solid Tumors

### Study Overview

A Phase 1, first-in-human, non-randomized open-label, adaptive-design outpatient study to assess the safety, tolerability, pharmacokinetics (PK), and pharmacodynamics (PD) of single and multiple doses of SON-1010 administered as subcutaneous (SC) doses to patients with advanced solid tumors every 14 to 28 days will be conducted. The study utilizes a traditional 3+3 dose-escalation design to establish the Recommended Phase 2 Dose (RP2D) and/or maximum tolerated dose (MTD) with up to 5 dose-escalation groups and expand the dataset at the recommended RP2D. A summary of the objectives and endpoints are presented in Table 2. The planned dosing escalation schedule for the 5 dose-escalation groups is provided in Table 3.

A total of approximately 36 patients will be enrolled: 6 patients at 5 dose level cohorts, with 1 to 6 patients in each cohort, for a total of up to 30 patients during dose escalation and an additional 6 (or 9 patients if only 3 were studied previously) patients at the RP2D and MTD (for a total of 12 at that dose) as an expansion group. A patient is considered enrolled if they agree to participate in the study after completion of the informed consent process and are accepted into the trial. Potential patients who are screened for the purpose of determining eligibility for the study, but do not participate in the study, are not considered enrolled, unless otherwise specified.

Patients receive SON-1010 in 14-28-day treatment cycles for up to 2 years until disease progression or discontinuation due to adverse events (AEs), although the study may be extended beyond that period based on patient factors, accumulated drug data, possible regulatory approvals for commercial use, and drug availability. If nonlinear PK are observed at any dose level, PK and PD samples from all prior cohorts may be used in an interim PK and PD analysis to select one or the other dose level escalation and timing, or intermediate doses may be included for up to 2 additional cohorts.

The RECIST v1.1 guidelines for measurable, nonmeasurable, target and nontarget lesions, and the objective tumor response criteria (complete response [CR], partial response [PR], stable disease [SD], or progressive disease) will be used to determine overall response rate at each dose level. Additionally, the serum and urine level of SON-1010 will be assessed with a commercial enzyme-linked immunosorbent assay for IL-12.

TABLE 2
Objectives and Endpoints

| | |
|---|---|
| Objectives | Endpoints |

Primary

| | |
|---|---|
| To evaluate the safety and tolerability of | Incidence of treatment-emergent adverse |
| SON-1010 | events (TEAEs), treatment-emergent serious |
| To establish the maximum tolerated dose | adverse events (SAEs), and changes from |
| (MTD) and/or recommended Phase 2 dose | baseline is safety parameters. |
| (RP2D) of SON-1010 | Incidence of dose-limiting toxicities (DLTs) |

Secondary

| | |
|---|---|
| To characterize the PK profile of SON-1010. | Serum PK parameters for a single-dose |
| To characterize the PD profile of SON-1010 | (Cmax, AUCO-last, AUCinf, tmax, t½, Kel, |
| To evaluate the potential for immunogenicity | Vz/F, CL/F) and multiple doses (Cmax, |
| of SON-1010 | AUCtrough, AUCO-last, AUCinf, tmax, t½, |
| | Kel, Vss/F, CLss/F) of SON-1010. |
| | Urine PK parameters (as above) of |
| | SON-1010. |
| | Serum measurements of SON-1010 PD |
| | markers (IFNγ, IL1B, IL2, IL4, IL6, IL8, |
| | IL10, and TNFα). |
| | Anti-drug antibodies determination. |

Exploratory

| | |
|---|---|
| To assess the antitumor activity of SON-1010 | Objective response rate per RECIST 1.1 and |
| To characterize the relationship between PK | iRECIST or other appropriate disease-specific |
| and PD in dosing of SON-1010 | response criteria. |
| | Determine the relationship between |
| | multiple-dose PK and PD |
| | (ie, exposure-response) of SON-1010. |

AE = adverse event;
AUCinf, AUCO-last, AUCtrough = area under the serum concentration versus time curve extrapolated to infinity from 0 to the last dose, and the trough levels;
CL/F and CL/F = apparent total body clearance;
Cmax = maximum serum concentration;
IL = interleukin;
IFNγ = interferon gamma;
iRECIST = immune Response Evaluation Criteria in Solid Tumors;
Kel = elimination rate constant;
ORR = objective response rate;
PD = pharmacodynamics;
PK = pharmacokinetics;
RECIST = response evaluation criteria in solid tumors, t½-terminal elimination half-life;
TNF = tumor necrosis factor alpha;
tmax = time to peak serum concentration;
Vz/F = apparent total volume of distribution
Note:
The PK assay will measure the IL-12 portion of SON-1010.

TABLE 3
Planned Dose Escalation Schedule

| Cohort | Dose Multiplier | Dose Escalation (μg/kg) | rhIL 12 ME (μg/kg) |
|---|---|---|---|
| M1 | — | 0.05 | 0.03 |
| M2 | 3x M1 | 0.15 | 0.10 |
| M3 | 2x M2 | 0.15 → 0.30 ª | 0.20 |
| M4 | 1.8x M3 | 0.15 → 0.54 | 0.36 |
| M5 | 1.4x M4 | 0.15 → 0.75 | 0.50 |

M1 = dose level 1;
ME = molar equivalent
^{a} A desensitizing dose of 0.15 μg/kg will be given on C1D1, with subsequent doses as indicated.

### Detailed Study Design

Once four baseline PK (and a single baseline PD) samples are obtained, the first patient in Cohort 1 receives the lowest dose of SON-1010 (0.05 μg/kg) planned as a sentinel patient for 1 week. A total of 3 patients were treated with that dose of SON-1010 and observed for at least 7 days after the 2nd dose (the dose limiting toxicity (DLT) observation period, consisting of cycle 1, day 1 (C1D1) to cycle 2, day 8 (C2D8)) before enrollment of the next cohort. In the event that a patient in any cohort experiences a DLT, the design included enrollment of up to 6 patients at that dose level.

For the first two 3-patient dose-escalation cohorts, 1 patient will be treated and observed for safety (as a sentinel subject) to cycle 1, day 5 (C1D5) before additional patients are treated in the cohort. Dose escalation to subsequent cohorts may proceed once all patients in a Cohort have received 2 doses of SON-1010 and have completed 35 days (the DLT observation period). Subsequent cohorts following dosing at M1 are planned at the doses shown in Table 3. Intermediate dose levels may be further expanded in consultation with the SRC but dose levels below M1 will not be evaluated.

If no DLTs are reported during the DLT period at any given dose level, accrual may advance to the next dose level. A cohort with 1 DLT during the DLT period will be expanded to include 6 patients. If DLTs occur in 2 or more patients in any cohort during the DLT period, then accrual at that dose will stop and a lower dose cohort will be expanded to 6 patients. This lower cohort dose level may be at the previous lower dose (if 6 patients have not already been accrued at that lower dose) or an intermediate dose level may be evaluated.

During dose escalation, additional patients may be enrolled at a dose previously cleared to explore further safety and tolerability, with approval of the Sponsor. Adverse events that occur in these patients will not be considered evaluable for DLT definition, however the SRC may consider these AEs when determining the RP2D for the expansion phase. Table 4 provides criteria for SON-1010 dose escalation.

TABLE 4
Criteria for SON-1010 Dose Escalation

| If a DLT is observed, | |
|---|---|
| regardless of dose in: | Response: |
| 0 of 3 patients with DLT | Proceed with enrollment in next cohort |
| 1 of 3 patients with DLT | Enroll up to 3 additional patients in |
| | current cohort |
| 1 of 6 patients with DLT | Proceed with enrollment in next cohort |
| ≥2 of 3 patients with DLT | Do not escalate; expand to 6 patients in |
| | previous cohort or at an intermediate |
| | dose level |
| ≥2 of 6 patients with DLT | Do not escalate; expand to 6 patients in |
| | previous cohort or at an intermediate |
| | dose level |

Note:
To accommodate logistics of enrollment at multiple centers, over-enrollment of a 3-patient cohort by an additional patient is allowed with approval of the Sponsor, provided no dose limiting toxicity has been observed in that cohort. All patients will be considered for dose limiting toxicity assessments

### Eligibility Criteria

Patients are eligible to be included in the study if all of the following criteria apply:
1. Age ≥18 at the time of informed consent
2. Must have histologically or cytologically verified solid epithelial or mesenchymal tumors.
3. Locally advanced or metastatic disease
4. Measurable disease by Response Evaluation Criteria in Solid Tumors (RECIST v1.1), or the modified criteria for immune-based therapeutics (termed iRECIST) as appropriate. Lesions situated in a previously irradiated area are considered measurable if progression has been demonstrated in such lesions.
5. Must have been treated with standard of care therapies for their disease and have no standard alternative treatment options that are deemed by the treating physician to offer reasonable or potentially better benefit, or not be a candidate for standard therapy for their disease due to an underlying physical condition. Note that if patients have alternative therapies available that are known to confer clinical benefit, they should be informed of these therapies during the informed consent process.
6. Must weigh >50 kg to ≤120 kg at screening (to facilitate SON-1010 dilution before dosing).
7. Eastern Cooperative Oncology Group (ECOG) performance status of 0 or 1
8. Adequate organ and bone marrow function, in the absence of growth factors, as defined by the following laboratory parameters by day-1:
a. Hematologic: neutrophils ≥1500/μL, platelets ≥100,000/μL, lymphocytes ≥500/μL, hemoglobin >9 g/dL (transfusion and/or erythropoietin not permitted within 2 weeks before blood draw)
b. Renal: estimated glomerular filtration rate ≥45 mL/min/1.73 m² (Levey 2009)
c. Hepatic: alanine aminotransferase (ALT) and aspartate aminotransferase (AST)≤2.5×ULN or ≤5×ULN if liver metastases; coagulation international normalized ratio (INR) and activated partial thromboplastin time (aPTT), ≤1.5×ULN; serum total bilirubin≤1.5×ULN or total bilirubin ≤ULN for patients with total bilirubin ≥1.5×ULN (except for elevated bilirubin secondary to Gilberts disease. Confirmation of Gilberts diagnosis requires: elevated unconjugated (indirect) bilirubin values; normal complete blood count (in previous 12 months), blood smear and reticulocyte count; normal transaminases and alkaline phosphatase in previous 12 months.
d. Chemistry: albumin ≥3.0 g/dL at screening
9. Females of childbearing potential, <1-year postmenopause who are not permanently sterile, must have a negative serum pregnancy test (beta-human chorionic gonadotropin [β-HCG]) at baseline (unless they have had a hysterectomy), and agree to use 2 highly effective methods of birth control during the study and for 30 days after the last dose of study intervention. Females who are not of childbearing potential (have had a tubal ligation, hysterectomy, or bilateral oophorectomy, or are >1-year postmenopause) or have a partner who has had a vasectomy do not need to use contraception. Nonchildbearing potential is defined as surgically sterile or postmenopausal (defined as 12 months of spontaneous amenorrhea). A follicle stimulating hormone (FSH) level >40 IU/L at screening will confirm postmenopausal status. If a patient is not sexually active, but becomes active, then she and her male partner must use 2 methods of adequate contraception.
10. Males and their female partners must use a highly effective method of birth control if female partner(s) is of childbearing potential and must not donate sperm during the study and for 30 days after the last dose of study intervention. Contraception guidance is described in Section 10.4.
11. Willing and able to provide signed informed consent as described in Section 10.1.3 which includes compliance with the requirements and restrictions listed in the informed consent form (ICF) and in this protocol.
12. Must be able to communicate well with Investigator and/or study site personnel and to comply with the requirements of the entire study.

### Exclusion Criteria

Patients are excluded from the study if any of the following criteria apply:
1. Known history of allergy to any component of study intervention.
2. History of severe allergic/anaphylactic reaction.
3. Infection with HIV-1 or HIV-2 or a history of Kaposi sarcoma and/or Multicentric Castleman Disease.
4. Current active liver disease from any cause, including hepatitis A (hepatitis A virus IgM positive), hepatitis B (hepatitis B virus [HBV] surface antigen positive), or hepatitis C (hepatitis C virus [HCV] antibody positive, confirmed by HCV ribonucleic acid). Patients with HCV with undetectable virus after treatment are eligible (note: patients must have completed curative antiviral therapy at least 4 weeks before screening).
5. Pregnancy and/or lactation
6. Has received a live or live-attenuated vaccine within 28 days before the first dose of study intervention. Note: Administration of killed vaccines and COVID-19 vaccines that are not live or live-attenuated are allowed if >14 days before the first dose.
7. History of any active infection requiring systemic antibiotics, antivirals or antifungals within 14 days before the first dose of study intervention, including COVID-19 as determined by the currently recommended testing strategy for acute infection.
8. Any acute noninfectious illness not resolved by 14 days before day 1.
9. History of or known or suspected autoimmune disease (exception(s): patients with vitiligo, Type I diabetes, resolved childhood atopic dermatitis, hypothyroidism, or hyperthyroidism that is clinically euthyroid at Screening are allowed). Other exceptions may be allowed following discussion with the Sponsor Medical Monitor for patients who have not received treatment for their autoimmune disorder in the past 3 years.
10. Known active central nervous system metastases and/or carcinomatous meningitis. Patients with previously treated brain metastases may participate provided they are clinically stable for at least 4 weeks before study entry and have no evidence of new or enlarging brain metastases.
11. Unresolved toxicities from prior anticancer therapy, defined as not resolved to baseline or to grade 1 by the CTCAE V 5.0 criteria (NCI 2017) except for alopecia, peripheral neuropathy, and hypothyroidism secondary to prior checkpoint inhibitor therapy if currently being treated and clinically euthyroid.
12. Receipt of any investigational agent or treatment within a period of 5 half-lives (or 28 days whichever is shorter) before the first dose of study intervention.
13. Any prior immunotherapy or treatment with checkpoint inhibitors, unless approved by the Sponsor, within a period of 5 half-lives (or 3 months, whichever is shorter) since the last dose of the therapy.
14. Use of systemic steroids >10 mg/day prednisone (or equivalent) within 14 days of enrollment, except for local (topical, nasal, or inhaled) steroid use. Limited doses of systemic steroids (e.g., in patients with exacerbation of reactive airway disease) must have been completed at least 10 days before enrollment. Steroid use to prevent IV contrast allergic reaction or anaphylaxis in patients who have known contrast allergies is allowed at any time before enrollment.
15. Active known second malignancy except any of the following:
   Adequately treated basal cell carcinoma, squamous cell carcinoma of the skin, or Stage 0 carcinoma in situ
   Adequately treated Stage I cancer from which the patient is currently in remission and has been in remission for >2 years
   Low-risk prostate cancer with Gleason score <7 and prostate-specific antigen <10 ng/mL
   Any other cancer from which the patient has been disease-free for >2 years
16. Use of biotin (ie Vitamin B7) or supplements containing biotin higher than the daily adequate intake of 30 μg (FDA 2019) (Note: patients who switch from a high dose to a dose of 30 μg/day or less are eligible).
17. Any of the following events within 6 months before baseline Day 1:
   Myocardial infarction
   Unstable angina
   Unstable symptomatic ischemic heart disease
   New York Heart Association class III or IV heart failure
   Thromboembolic events (e.g., pericardial effusion, restrictive cardiomyopathy, severe untreated valvular stenosis, or severe congenital heart disease)
18. Electrocardiogram QT interval corrected for heart rate (QTc)>470 msec, measured by Fridericia's formula [QTcF=QT/(RR0.33)]. If the QTc is prolonged in a patient with a pacemaker or bundle branch block, the patient may be enrolled in the study if confirmed by the medical monitor.

### Preliminary Results

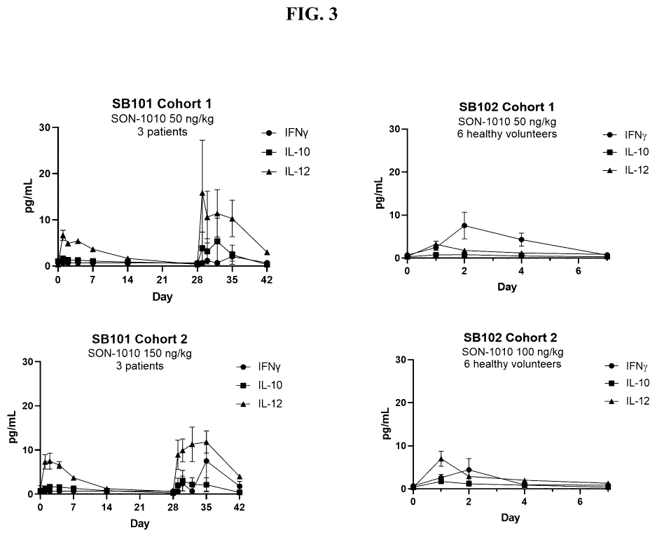

Preliminary results depicting the cytokine response levels for individuals in Cohort 1 and 2 are providing in FIG. 3. Acute inflammation was assessed with a Luminex bead assay for multiple analytes in studies SB101 and SB102. IL-12p70, which is used to measure SON-1010, showed extended pharmacokinetics, and a controlled, dose-related increase in IFNγ was seen. There was no indication of cytokine release syndrome (CRS). While there was also minimal to no signal for IL-1β, IL-6, and IL-8, IL-10 was induced at a low level.

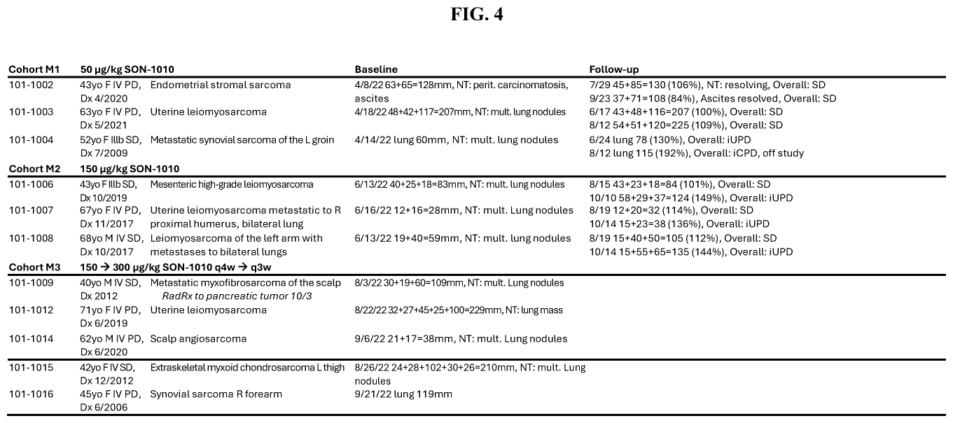

Additionally, FIG. 4 provides preliminary efficacy results for Cohort M1, M2, and M3. One patient is stable and showing resolution of target tumors and ascites. Two patients who had PD at entry have SD.

### Example 2: A Proof-of-Concept Study to Assess the Combination of SON-1010 (IL-12-FHAB) and an Anti-PD-L1 Antibody (Zimberelimab) in Patients with Platinum-Resistant Ovarian Cancer

### Study Overview

A Phase 1b/2a multicenter, dose-escalation study to assess the safety, tolerability, PD, and efficacy of SON-1010 administered subcutaneously (SC) alone or in combination with an anti-PD-L1 antibody (zimberelimab) administered intravenously (IV) will be conducted in two parts. Part 1 will utilize a traditional 3+3 dose-escalation design to establish the Recommended Phase 2 Dose (RP2D) and/or maximum tolerated dose (MTD) of a combination of SON-1010 and zimberelimab in patients with advanced solid tumors with up to 5 dose-escalation groups and to expand the dataset at the recommended RP2D. Part 2 will use the MTD as one of three randomized groups to assess the efficacy of the combination over SON-1010 alone or the standard of care (SOC) with the aim of establishing proof-of-concept (POC) in patients with platinum-resistant ovarian cancer (PROC).

The MTD is defined as the highest dose of SON-1010 that does not cause 2 or more DLTs in a 6-patient cohort. The RP2D will be the MTD unless: significant clinical anti-tumor effect (such as complete response [CR], partial response [PR], or stable disease for >4 months) is non-linear or seen below the MTD, in which case a clinically active dose level may be selected as an RP2D; or toxicities observed beyond Cycle 1 require reducing the RP2D below the MTD level; or MTD is not achieved, in which case the highest dose level administered may become an RP2D, or consideration may be given to the lowest dose level that achieves maximal pharmacodynamic activity.

The dosing strategy for this study will utilize a desensitizing 1^{st} dose of SON-1010, preferably a dose 150 ng/kg, followed by a potentially higher maintenance dose to minimize toxicity and improve the chance of reaching a therapeutic dose of SON-1010. A summary of the objectives and endpoints are presented in Table 5.

The Part 1 data will be summarized by dose cohort, expansion cohort, and total patient populations. All assessments before the first dose of study drug will be considered as baseline. If there are multiple baseline assessments, the most recent one will be used for statistical analysis. No formal hypothesis testing is planned for safety or PD parameters.

Part 2 of the study will use an automated central randomization procedure to allocate patients in a 1:1:1 ratio to each of the three treatment groups. The randomization will be stratified based on:
Stage of disease at diagnosis and debulking status
Stage III and maximal residual lesion diameter≤1 cm
Stage III and maximal residual lesion diameter >1 cm
Stage IV
Determination of whether a subject is experiencing
A first or second relapse
A third or greater relapse

TABLE 5
Objectives and Endpoints

| | |
|---|---|
| Objectives | Endpoints |

Primary

| | |
|---|---|
| To evaluate the safety and tolerability of | Incidence of treatment-emergent adverse |
| SON-1010 in combination with zimberelimab | events (TEAEs) and treatment-emergent |
| To establish the maximum tolerated dose | serious adverse events (SAEs) and changes |
| (MTD) and/or recommended Phase 2 dose | from baseline in safety parameters. |
| (RP2D) of SON-1010 dosed with | Incidence of dose-limiting toxicities (DLTs) |
| zimberelimab | |

Secondary

| | |
|---|---|
| To assess the anti-tumor activity of SON- | RECIST v1.1 or iRECIST assessment of: |
| 1010 dosed with zimberelimab compared with | Progression-free survival (PFS) |
| SON-1010 alone | Overall survival (OS) |
| | Best overall response rate (CR+PR), and |
| | duration of response (DOR) |
| | Disease control rate (CR + PR + SD + |
| | non-CR/non-PD) |
| | Cancer Antigen (CA)125 response or |
| | progression (Gynecologic Cancer InterGroup |
| | [GCIG] criteria) |
| | Progression free rate (PFR) at 6 months. |

Exploratory

| | |
|---|---|
| To characterize the PD profile of SON-1010 | Single- and multiple-dose serum PD |
| dosed with zimberelimab compared with | parameters of inflammation (IFNγ, IL1B, IL2, |
| SON-1010 alone | IL4, IL6, IL8, IL10, IL12p70, IL13, and |
| | TNFα). |

AE = adverse event;
CR = complete response;
IL = interleukin;
IFNγ = interferon gamma;
iRECIST = immune response evaluation criteria in solid tumors;
PD = pharmacodynamics or progressive disease;
PR = partial response;
SD = stable disease;
TNFα = tumor necrosis factor alpha

### Part 1: Dose Escalation

Dose escalation will be done in Part 1 in patients with advanced solid tumors based on the planned dose escalation schedule provided in Table 6. A total of approximately 36 patients (30 patients during dose escalation, plus 6 patients during RP2D/MTD expansion) will be included. Up to 12 additional patients in up to 2 intermediate dose level Cohorts may be added.

The first (sentinel) patient in Cohort C1 will receive a desensitizing dose of SON-1010 in combination with a fixed dose of zimberelimab (360 mg), followed by the SON-1010 maintenance dose with zimberelimab every 3 weeks. The patient will be treated and observed for 7 days before enrollment of an additional patient in that cohort. Each cohort will be treated and observed for at least 28 days (the dose limiting toxicity (DLT) observation period) consisting of cycle 1, day 1 (C1D1) to cycle 2, day 8 (C2D8). A Trial Steering Committee (TSC) will then meet to discuss the results of each cohort in Part 1 before enrollment of the next cohort. Efficacy will be followed by CA125 levels (Rustin 2011) and RECIST v1. 1 scores (Eisenhauer 2009) every 2 months until progression or withdrawal criteria are met. If the patient is clinically stable and the PI chooses to continue, iRECIST rules can be followed until progression is confirmed (Seymour 2017).

TABLE 6
Planned SON-1010 Dose Escalation Schedule in Part 1

| Cohort | SON-1010 Dose (μg/kg) ^{a} | rhIL-12 ME (μg/kg) |
|---|---|---|
| C1 | 0.050/0.100 | 0.033/0.066 |
| C2 | 0.100/0.150 | 0.066/0.100 |
| C3 | 0.150/0.150 | 0.100/0.100 |
| C4 | 0.150/0.300 | 0.100/0.200 |
| C5 | 0.150/0.450 | 0.100/0.300 |

ME = molar equivalent
^{a} Desensitizing dose/Maintenance dose.

If no DLTs are reported during the DLT period at a given dose level, accrual may advance to the next dose level. A cohort with 1 DLT during the DLT period will be expanded to 6 patients. If DLTs occur in 2 or more patients in any cohort during the DLT period, then accrual at that dose will stop and a lower dose cohort will be expanded to 6 patients. This lower cohort dose level may be at the previous lower dose (if 6 patients have not already been accrued at that lower dose) or an intermediate dose level may be evaluated. If fewer than 2 DLTs are observed in the 6 patients at this lower dose, this lower dose will be considered the MTD and the most effective cohort will be expanded to 12 patients to confirm the RP2D.

Dose escalation to subsequent cohorts is based on the described dose-escalation schema in Table 7 and it may proceed once all patients in a Cohort have received 2 doses of the combination and have completed 28 days. Subsequent cohorts following dosing at Cl are planned at the doses shown in Table 6. During dose escalation, additional patients may be enrolled at a dose previously cleared to explore further safety and tolerability, with approval of the Sponsor. Adverse events that occur in these patients will not be considered evaluable for DLT definition, however the TSC may consider these AEs when determining the RP2D for the expansion phase.

TABLE 7
Decision Schema for SON-1010 Phase 1 Dose Escalation

| For any given dose level, | |
|---|---|
| If a DLT is observed in: | Then: |
| 0 in 3 patients* | Proceed with enrollment in next cohort |
| 1 of 3 patients | Enroll up to 3 additional patients in |
| | current cohort |
| 1 of 6 patients | Proceed with enrollment in next cohort |
| ≥2 of 6 patients | Do not escalate; enroll up to 3 additional |
| | patients in previous cohort or at an |
| | intermediate dose level |
| ≥2 of 3 patients | Do not escalate; enroll up to 3 additional |
| | patients in previous cohort or at an |
| | intermediate dose level |

SON-1010 will be administered as a single SC dose with zimberelimab at a fixed dose of 360 mg IV in each 21-day treatment cycle (up to 35 cycles). Patients will receive SON-1010 and zimberelimab at the cohort dose level until disease progression or discontinuation due to AEs. Up to 5 dose level cohorts are planned with 3 to 6 patients in each cohort for a total of about 30 patients during dose escalation (Table 6). Enrollment of 6-9 patients at the RP2D/MTD (for a total of 12 at that dose) is also planned as an expansion group.

Samples for PD will be taken at several timepoints during the first two cycles in Part 1 of this study to evaluate the presence and effect of SON-1010 after single and multiple doses. Primary PD parameters will include IFNγ, IL1β, IL2, IL4, IL6, IL8, IL10, IL 12p70, IL13, and TNFα.

### Part 2: Proof of Concept

Once the RP2D of SON-1010 in combination with zimberelimab has been determined in Part 1, patients with PROC can be randomized 1:1:1 into one of 3 groups (R1 to R3) in Part 2.
R1: SON-1010 alone at the RP2D dose of SON-1010 from Example 1.
R2: SON-1010 at the RP2D dose of SON-1010 from Part 1 in combination with zimberelimab
R3: Standard of Care (PI decision regarding chemotherapy selection).

Approximately 40 patients with recurrent PROC will be included in each group for a total of 120 patients. SON-1010 will be administered as a single SC dose with zimberelimab at a fixed dose of 360 mg IV in each 21-day treatment cycle (up to 35 cycles) in Groups C1 to C5, R1 and R2. The SC dose will be injected in alternating upper arms over the deltoid muscle, or, if prior surgery precludes the use of an arm, injections may be done in the thigh (preferred) or abdomen. Zimberelimab will be dosed IV every 21 days in Group R2. Patients in Group R3 will be dosed according to the SOC selected by the PI.

The duration of study participation is anticipated to be approximately 7 months on average including the screening period, assuming an average of 8 doses per patient. Treatment will be supported for up to 2 years, but may be extended beyond that period based on patient factors, accumulated drug data, possible regulatory approvals for commercial use, and drug availability. The primary clinical efficacy endpoint in Part 2 is PFS (inclusive of any death), where disease progression is primarily determined per RECIST v1.1 criteria.

### Eligibility Criteria

Individuals eligible to participate in the study must meet the following criteria:
1. Age ≥18 years at the time of informed consent
2. Part 1: Must have histologically or cytologically verified solid tumors and patients must have locally advanced or metastatic disease. Must have been treated with standard of care therapies for their disease and have no standard alternative treatment options that are deemed by the treating physician to offer reasonable or potentially better benefit. Part 2: Must have platinum-resistant ovarian cancer (defined as recurrence within 6-mo following at least partial response to and completion of a platinum-containing regimen), including epithelial, fallopian tube, or 1° peritoneal carcinoma. Subjects with platinum-refractory ovarian cancer are eligible, provided the outcome was to a 2^{nd} line or later, repeated platinum regimen (i.e. not during 1^{st} line). Refractory is defined as disease that failed to achieve at least a PR to a platinum-containing regimen (i.e., SD or actual disease progression). Subjects may have had one or more alternative regimen(s) before this trial, including maintenance therapy between consecutive lines of therapy.
3. Part 2: Histologic documentation of carcinoma is required and the following subtypes are eligible: high grade (grade ≥3+) serous carcinoma, low-grade serous carcinoma, endometrioid, clear cell, or mucinous ovarian adenocarcinoma, poorly-differentiated adenocarcinoma, or mixed (including above subtypes). Note that synchronous serous or endometrioid uterine or fallopian cancers are allowed.
4. Locally advanced or metastatic disease that is measurable by the Response Evaluation Criteria in Solid Tumors (RECIST v1.1) or (for PROC) a CA125 level that is >2-fold above the ULN. Lesions situated in a previously irradiated area are considered measurable if progression has been demonstrated in such lesions.
5. Must weigh >50 kg and ≤120 kg at screening (to facilitate SON-1010 dilution before dosing).
6. Eastern Cooperative Oncology Group (ECOG) performance status of 0 or 1.
7. Adequate organ and bone marrow function, in the absence of growth factors, as defined by the following laboratory parameters:

| | |
|---|---|
| System | Laboratory Value |

Hematological:

| | |
|---|---|
| Neutrophils | ≥1500/μL |
| Platelets a | ≥100,000/μL |
| Hemoglobin a | ≥9 g/μL |
| Lymphocytes | ≥500/μL |

Renal:

| | |
|---|---|
| eGFR (CKD-EPI creatinine calculation) | ≥45 mL/min/1.73 m2 |
| (Levey 2009) | |

Hepatic:

| | |
|---|---|
| Serum total bilirubin | ≤1.5 × ULN or |
| Direct bilirubin | ≤ ULN for patients with total bilirubin |
| | >1.5 × ULN |

Exception for elevated bilirubin secondary to Gilberts disease. Confirmation of Gilberts diagnosis requires
elevated unconjugated (indirect) bilirubin values, normal complete
blood count (in previous 12 months), normal blood smear and reticulocyte count, normal
transaminases and alkaline phosphatase (in previous 12 months).

| | |
|---|---|
| AST/SGOT and ALT/SGPT | <2.5 × ULN or ≤5 × ULN if liver |
| | metastases |

Coagulation (for those patients not receiving anticoagulants):

| | |
|---|---|
| INR | ≤1.5 × ULN |
| aPTT | ≤1.5 × ULN |

Chemistry:

| | |
|---|---|
| Albumin | >3.0 g/dL at screening |

ALT = alanine aminotransferase;
aPTT = activated partial thromboplastin time; AST = aspartate amino-transferase;
CKD-EPI = Chronic Kidney Disease-Epidemiology Collaboration;
eGFR = estimated glomerular filtration rate;
INR = international normalized ratio;
SGOT = serum glutamic oxaloacetic transaminase;
SGPT = serum glutamic pyruvic transaminase;
ULN = upper limit of normal a Transfusion and/or erythropoietin not permitted within 2 weeks prior to blood draw

8. Females of childbearing potential, <1-year postmenopause who are not permanently sterile, must have a negative serum pregnancy test (beta-human chorionic gonadotropin [β-HCG]) at baseline, and agree to use 2 highly effective methods of birth control during the study and for 30 days after the last dose of study intervention. Females who are not of childbearing potential (have had a tubal ligation, hysterectomy, or bilateral oophorectomy, or are ≥1 year postmenopause) or have a partner who has had a vasectomy do not need to use any contraception. Adequate methods of contraception for participants include:
   Condom with spermicidal gel
   Diaphragm with spermicidal gel
   Coil (intrauterine device)
   Oral contraceptive pill
   Depot progesterone injections
   Progesterone implant (eg, Implanon®)
   NuvaRing®
   Ortho Evra®

Nonchildbearing potential is defined as surgically sterile (documented hysterectomy, tubal ligation, or bilateral salpingo-oophorectomy) or postmenopausal (defined as 12 months of spontaneous amenorrhea). A follicle stimulating hormone (FSH) level >35 IU/L at screening will be performed to confirm status. If a participant is not sexually active, but becomes active, then she and her male partner must use 2 methods of adequate contraception.
9. Males and their female partners must use a highly effective method of birth control if female partner(s) is of childbearing potential and must not donate sperm during the study and for 30 days after the last dose of study intervention.
10. Willing and able to provide signed informed consent, which includes compliance with the requirements and restrictions listed in the informed consent form (ICF) and in this protocol.

### Exclusion Criteria

Individuals who meet any of the following exclusion criteria will not be eligible to participate in this study:
1. Known history of allergy to any component of study intervention or a history of severe allergic/anaphylactic reaction.
2. Hospitalization for subacute bowel obstruction within 28 days prior to CID1.
3. Infection with HIV-1 or HIV-2 with a history of Kaposi sarcoma and/or Multicentric Castleman Disease.
4. Current active liver disease from any cause, including hepatitis A (hepatitis A virus IgM positive), hepatitis B (hepatitis B virus [HBV] surface antigen positive), or hepatitis C (hepatitis C virus [HCV] antibody positive, confirmed by HCV ribonucleic acid). Patients with HCV with undetectable virus after treatment are eligible (note: patients must have completed curative anti-viral therapy at least 4 weeks prior to screening). Patients with a prior history of HBV are eligible if quantitative PCR for HBV DNA is negative (note: patients must have received HBV antiviral therapy for at least 4 weeks prior to screening)
5. Pregnancy and/or lactation
6. Has received a live or live-attenuated vaccine within 30 days prior to the first dose of study intervention. (Note: Administration of killed vaccines and COVID-19 vaccines that are not live or live-attenuated are allowed if >14 days.)
7. History of any active infection requiring systemic antibiotics, antivirals or antifungals, including COVID-19, within 14 days before the first dose of study intervention.
8. Any acute noninfectious illness not resolved by 14 days before day 1.
9. History of or known or suspected autoimmune disease (exception(s): patients with vitiligo, Type I diabetes, resolved childhood atopic dermatitis, hypothyroidism, or hyperthyroidism that is clinically euthyroid at Screening are allowed). Other exceptions may be allowed following discussion with the Sponsor Medical Monitor for patients who have not received treatment for their autoimmune disorder in the past 3 years
10. Known active central nervous system metastases and/or carcinomatous meningitis. Patients with previously treated brain metastases may participate provided they are clinically stable for at least 4 weeks prior to study entry and have no evidence of new or enlarging brain metastases.
11. Unresolved toxicities from prior anticancer therapy, defined as not resolved to baseline or to grade 1 (NCI 2017), except for alopecia, peripheral neuropathy, and hypothyroidism secondary to prior checkpoint inhibitor therapy if currently being treated and clinically euthyroid.
12. Receipt of any investigational agent or treatment within 21 days or 5 half-lives, whichever is shorter, before the first dose of study intervention.
13. Any prior immunotherapy or treatment with checkpoint inhibitors, unless approved by the Sponsor, within a period of 5 half-lives (or 3 months, whichever is shorter) since the last dose of the therapy.
14. Use of systemic steroids >10 mg/day prednisone (or equivalent) within 10 days of enrollment, except for local (topical, nasal, or inhaled) steroid use. Limited doses of systemic steroids (eg, in patients with exacerbation of reactive airway disease) must have completed at least 10 days before enrollment. Steroid use to prevent IV contrast allergic reaction or anaphylaxis in patients who have known contrast allergies is allowed at any time before enrollment.
15. Active known second malignancy with the exception of any of the following:
   Adequately treated basal cell carcinoma, squamous cell carcinoma of the skin, or in situ cervical cancer
   Adequately treated Stage I cancer from which the patient is currently in remission and has been in remission for >2 years;
   Low-risk prostate cancer with Gleason score <7 and prostate-specific antigen <10 ng/ml; or
   Any other cancer from which the patient has been disease-free for >2 years.
16. Use of biotin (i.e., Vitamin B7) or supplements containing biotin higher than the daily adequate intake of 30 μg (FDA 2019) (Note: Patients who switch from a high dose to a dose of 30 μg/day or less are eligible for study entry)
17. Any of the following within 6 months before Baseline Day 1:
   Myocardial infarction;
   Unstable angina;
   Unstable symptomatic ischemic heart disease;
   New York Heart Association (NYHA) class III or IV heart failure;
   Thromboembolic events (e.g., deep vein thrombosis, pulmonary embolism, or symptomatic cerebrovascular events);
   Any other significant cardiac condition (e.g., pericardial effusion, restrictive cardiomyopathy, severe untreated valvular stenosis, or severe congenital heart disease);
   ECG QT interval corrected for heart rate (QTc)>470 msec, measured by Fridericia's formula [QTcF=QT/(RR\hat{ }0.33)]. If the QTc is prolonged in a patient with a pacemaker or bundle branch block, the patient may be enrolled in the study if confirmed by the medical monitor.

Specific embodiments disclosed herein can be further limited in the claims using “consisting of” or “consisting essentially of” language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the disclosure so claimed are inherently or expressly described and enabled herein.

In cases where numerical values are indicated in the context of the present disclosure, the skilled person will understand that the technical effect of the feature in question is ensured within an interval of accuracy, which typically encompasses a deviation of the numerical value given of ±10%, and preferably of ±5%. At the very least, and not as an attempt to limit the application of the doctrine of equivalents to the scope of the claims, each numerical parameter should at least be construed in light of the number of reported significant digits and by applying ordinary rounding techniques.

Unless otherwise indicated, all numbers expressing quantities of ingredients, properties such as molecular weight and median size, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure.

The terms “a,” “an,” “the” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is deemed to contain the group as modified, thus fulfilling the written description of all Markush groups used in the appended claims.

Certain embodiments of this disclosure are described herein, including the best mode known to the inventor for carrying out the disclosure. Of course, variations on these described embodiments will become apparent to those of ordinary skill in the art upon reading the foregoing description. The inventor expects skilled artisans to employ such variations as appropriate, and the inventor intends for the disclosure to be practiced other than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

It is to be understood that the embodiments of the disclosure disclosed herein are illustrative of the principles of the present disclosure. Other modifications that can be employed are within the scope of the disclosure. Thus, by way of example, but not of limitation, alternative configurations of the present disclosure can be utilized in accordance with the teachings herein. Accordingly, the present disclosure is not limited to that precisely as shown and described.

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary only, with the true scope and spirit of the disclosure being indicated by the following claims. All references, patents, and patent applications referred to in this application are herein incorporated by reference in their entirety.

1. A method for treating a cancer in a subject in need thereof, the method comprising: administering to the subject in need thereof an effective first dose of an IL-12 albumin binding domain (ABD) fusion protein and an effective second dose of the IL-12 ABD fusion protein every three weeks or four weeks after the first dose, wherein the second dose is greater than the first dose.

2. The method of claim 1, wherein the effective second and subsequent doses of the IL-12 ABD fusion protein is administered every three weeks or every four weeks after the first dose.

3. (canceled)

4. The method of claim 1, wherein the first dose is about 25 ng/kg to about 750 ng/kg.

5. (canceled)

6. The method of claim 1, wherein the second and subsequent doses are about 50 ng/kg to about 1.5 μg/kg.

7-9. (canceled)

10. The method of claim 1, wherein the first dose amount and the second and subsequent dose amounts are administered intravenously or subcutaneously.

11. (canceled)

12. The method of claim 1, wherein the albumin binding domain of the IL-12 ABD fusion protein comprises a variable heavy domain comprising SEQ ID NO:2 or a variant thereof, and a variable light domain comprising SEQ ID NO:6 or a variant thereof.

13. The method of claim 12, wherein the albumin binding domain is an scFv that comprises the amino acid sequence of SEQ ID NO: 1 or a variant thereof.

14. The method of claim 13, wherein the IL-12 of the IL-12 fusion protein is a single chain IL-12 comprising a p35 subunit covalently attached to a p40 subunit.

15. The method of claim 14, wherein the p40 subunit has the amino acid sequence of SEQ ID NO: 12 or a variant thereof, and the p35 subunit has the amino acid sequence of SEQ ID NO:13 or a variant thereof.

16. (canceled)

17. The method of claim 14, wherein the single chain IL-12 has the amino acid sequence of SEQ ID NO:10.

18. The method of claim 12, wherein the IL-12 ABD fusion protein has the amino acid sequence of SEQ ID NO: 11.

19-20. (canceled)

21. The method of claim 1, wherein the cancer is a solid epithelial or mesenchymal tumor.

22. (canceled)

23. The method of claim 1 further comprising the step of administering a checkpoint inhibitor to the subject in need thereof.

24. (canceled)

25. A method of achieving a clinical response or a partial clinical response of a solid tumor in a human patient, the method comprising:
administering to the human patient an effective first dose of an IL-12 albumin binding domain (ABD) fusion protein and an effective second dose of the IL-12 ABD fusion protein every two weeks, three weeks, or four weeks after the first dose, wherein the second dose is greater than the first dose.

26. The method of claim 25, wherein the effective second and subsequent doses of the IL-12 ABD fusion protein are administered every three weeks or four weeks after the first dose.

27. (canceled)

28. The method of claim 25, wherein the first dose is about 25 ng/kg to about 750 ng/kg.

29. (canceled)

30. The method of claim 25, wherein the second and subsequent doses are about 50 ng/kg to about 1.5 μg/kg.

31-33. (canceled)

34. The method of claim 25, wherein the first dose amount and the second and subsequent dose amounts are administered intravenously or subcutaneously.

35. (canceled)

36. The method of claim 25, wherein the albumin binding domain of the IL-12 ABD fusion protein comprises a variable heavy domain comprising SEQ ID NO:2 or a variant thereof, and a variable light domain comprising SEQ ID NO:6 or a variant thereof.

37. The method of claim 36, wherein the albumin binding domain is an scFv that comprises the amino acid sequence of SEQ ID NO: 1 or a variant thereof.

38. The method of claim 37, wherein the IL-12 of the IL-12 fusion protein is a single chain IL-12 comprising a p35 subunit covalently attached to a p40 subunit.

39. The method of claim 38, wherein the p40 subunit has the amino acid sequence of SEQ ID NO: 12 or a variant thereof, and the p35 subunit has the amino acid sequence of SEQ ID NO: 13 or a variant thereof.

40. (canceled)

41. The method of claim 38, wherein the single chain IL-12 has the amino acid sequence of SEQ ID NO: 10.

42. The method of claim 36, wherein the IL-12 ABD fusion protein has the amino acid sequence of SEQ ID NO:11.

43. The method of claim 25, wherein the solid tumor is a solid epithelial or mesenchymal tumor.

44. The method of claim 25 further comprising the step of administering a checkpoint inhibitor to the subject in need thereof.
